(19) United States
(12) Patent Application Publication
(10) Pub. No.: US 2023/0380677 A1
(43) Pub. Date: Nov. 30, 2023

(54) KITS FOR MINIMALLY INVASIVE PERCUTANEOUS NEPHROLITHOTOMY (PCNL) AND SIALENDOSCOPY

(71) Applicant: MicronVision Corp., Bellevue, WA (US)

(72) Inventor: Xiaolong OuYang, Bellevue, WA (US)

(21) Appl. No.: 18/233,282

(22) Filed: Aug. 11, 2023

Related U.S. Application Data
(63) Continuation-in-part of application No. 17/720,143, filed on Apr. 13, 2022, now Pat. No. 11,771,304.
Application No. 17/720,143 is a continuation-in-part of application No. 17/521,397, filed on Nov. 8, 2021.
Application No. 17/521,397 is a continuation-in-part of application No. 17/370,575, filed on Jul. 8, 2021.
(60) Provisional application No. 63/522,395, filed on Jun. 21, 2023.
(60) Provisional application No. 63/521,704, filed on Jun. 19, 2023.

(51) Int. Cl. A61B 1/307; A61B 1/00; A61B 17/34; A61B 17/00; A61B 1/06; A61B 1/07; A61B 1/005; A61B 1/018

(52) U.S. Cl. CPC A61B 1/307; A61B 1/00103; A61B 1/00045; A61B 17/3468; A61B 17/00234; A61B 1/0684; A61B 1/07; A61B 1/0051; A61B 1/018; A61B 2017/00362; A61B 2017/00238; A61B 46/10

(57) ABSTRACT

A kit comprising a first single-use portion releasably coupled with a reusable portion for use in a first stage of a PCNL medical procedure to penetrate to a patient's kidney and/or ureter and view on an electronic display the work area and a guidewire passed through the first single-use portion, and a second single-use portion releasable coupled with said reusable portion for use in a second stage of a PCNL medical procedure, after the first single-use portion has been withdrawn from the patient, to view on the electronic display the work area and surgical tools treating the area during and after completion of treatment. Another kit comprises a single-use device for sialendoscopy.

### REFERENCE TO RELATED APPLICATIONS

This application claims the benefit of provisional applications Ser. Nos. 63/522,395 filed Jun. 21, 2023 and 63/521,704 filed Jun. 19, 2023, and is a continuation in-part of each of parent applications: (a) Ser. No. 17/720,143 filed Apr. 13, 2022, (b) Ser. No. 17/521,397 filed Nov. 8, 2021, and (c) Ser. No. 17/370,575 filed Jul. 8, 2021.

This application incorporates by reference the entirety of the foregoing patent applications and claims the benefit of the filing date of each as well as of the applications that they incorporate by reference, directly or indirectly and the benefit of which they claim, including U.S. provisional applications, U.S. non-provisional applications, and international applications.

### FIELD

This patent specification generally relates to portable, hand-held endoscopes that are entirely or partly single-use.

### BACKGROUND

Percutaneous nephrolithotomy (PCNL) is a medical procedure to remove stones or perform other surgery on a patient's kidney and/or ureter. In some examples, a retrograde pyelogram is done to locate the stone. Contrast is introduced slowly under fluoroscopy monitoring to observe a sequence of calyces being filled, to help identify the position of posterior calyx. Then, in a first stage of the procedure a percutaneous nephrolithotomy (PCN) needle is passed through the skin and soft tissue into the pelvis of the kidney. The position of the needle is confirmed with an imaging modality such as fluoroscopy or ultrasound. A guide wire is passed through the needle into the pelvis. The needle is then withdrawn, with the guide wire remaining in the pelvis. One or more dilators are passed over the guide wire and then a working sheath is introduced. A nephroscope is then passed inside and stones are taken out, possibly after first being crushed, or another medical procedure is performed on the kidney or ureter. The nephroscope is an optical instrument which allows one to inspect the renal cavitary system and to perform different therapeutic procedures under direct visual control. It contains the optical system for observing the working area and a working channel located in the nephroscope to enable insertion of a lithothripter or different working elements (stone forceps, extraction probe, etc.). The same axial channel also enables irrigation fluid flow. The fluid returns through space between the nephroscope and its sheath. The surgery can take 3-4 hours. A conventional nephroscope is not single-use, which requires thorough decontamination between patients and shortens the instrument's life. Known nephroscopes use an optical path for viewing the working area, which requires the surgeon to view the working area through an eyepiece attached to the nephoscope and thus limits the surgeon's body and head positions as well as the viewing angle.

Sialendoscopy is a minimally invasive procedure to diagnose and treat salivary gland disorders including stones, strictures, chronic inflammation and other problems affecting the major salivary glands. Reusable instruments for have been offered by Karl Storz of Tuttlingen, Germany and other companies and can be flexible, semiflexible, or rigid, and can be only for viewing and diagnosis or can include working channels for surgical instruments. Such known instruments are not single-use and therefore need to be thoroughly decontaminated between patients, which shortens their life, requires the expense of additional personnel and equipment, and still cannot completely eliminate the risk of cross-contamination. Known instruments are discussed in Iro H, Zenk J, Koch M, and Bozzato A, The Erlangen Salivary Gland Project, Part 1, Endo Press GmbH, Erlangen, Germany, 2005 (print 2007), ISBN 978-3-89756-149-6, which is hereby incorporated by reference.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY

As described in the initially presented claims but subject to amendments thereof during prosecuting this patent application, according to some embodiments a kit for PCNL medical procedures contains two different single-use portions that are utilized in respective different stages of the medical procedure. In each stage a reusable portion is coupled with the single-use portion to enable viewing an electronic image of the work area. Contamination risks are greatly reduced, the expense of time and personnel and equipment for decontaminating a reusable nephroscope are eliminated, and viewing the electronic image enables greater freedom of movement of the surgeon and reduces fatigue.

In addition, after the needle is inserted under guidance of fluoroscopy or ultrasound, it is very valuable to have optical vision at the tip of the needle to visually confirm the targeted location and embodiments disclosed herein accomplish this without an additional insertion of a nephoscope or a tool for inserting a guidewire.

According to some embodiments, a kit for performing minimally invasive percutaneous nephrolithotomy (PCNL) medical procedure comprises: a first single-use portion comprising a first handle, a first cannula that extends distally from the first handle and has a sharpened distal end, a first channel that extends from a first proximal port in the handle to a first distal port in the cannula and is configured for insertion of guidewire through and distally out of the first channel, and a first imaging module that is at a distal end of the first cannula and comprises a first light source and a first image sensor; a second single-use portion comprising a hub, a second cannula that extends distally from the hub, and a second imaging module that is a distal end of the second cannula and comprises a second light source and a second image sensor; wherein the first cannula is configured for insertion to or inoto a patient's kidney and/or ureter and the first imaging module is configured to take first images thereof that selectively include any guidewire portion extending out of the distal end of the first channel during a first stage of the medical procedure; wherein the second cannula is configured for insertion to or into the patient's kidney and/or ureter during a second stage of the medical procedure, after the first canula has been withdrawn from the patient, and the second imaging module is configured to take second images thereof during a second stage of the medical procedure; a reusable portion that comprises a second handle, a display carried thereby and image-processing electronics; wherein the reusable portion is configured to couple with the first single-use portion during said first stage of the medical procedure to thereby receive said first images and process and display them, and to couple with said second single-use portion during said second stage of the medical procedure to thereby receive said second images and process and display them; wherein said kit further includes a first pathway configured to convey said first images from the first imaging module to the reusable portion during the first stage of the medical procedure and a second pathway configured to convey the second images from the second imaging module to the reusable portion during in second stage of the medical procedure.

According to some embodiments, the kit can further comprise one or more of the following: (a) a sterile package enclosing said first and second single-use portions for shipping to a user and for storing until needed for a PCNL procedure; (b) the second cannula can include a flexible distal portion configured to bend away from a long axis and the second cannula and the reusable portion can include an electric motor operatively coupled with said flexible portion to bend the flexible portion in a selected direction and through a selected angle under manual control over the motor operation; (c) the reusable portion can have a side opening with a shaft extending transversely to the long axis and configured to be rotated by said motor, and said hub can include a gear mating with said shaft during said second stage of the medical procedure; (d) the hub can include a finger control operatively coupled with said flexible portion and configured to bend the flexible portion in a selected direction and through a selected angle in response to force exerted on the finger control; (e) the first single-use portion can be configured to be withdrawn over the guidewire after the first stage of the medical procedure, leaving the guidewire in the patient, and further including a trocar configured to the inserted in the patient to the patient's kidney over the guidewire, enabling surgical instruments to be inserted through the trocar to the kidney and/or ureter; (f) said first pathway can comprise a cable operatively coupled with said first imaging module, extending from the proximal end of the first handle and terminating in a first connector and the reusable portion can comprise a matching connector configured to make electrical contact with the first connector and to transmit power from the reusable portion to the first single-use portion and to convey said first images to the reusable portion; (g) said first pathway can comprise a wireless connection between said first imaging module and said reusable portion configured to convey said first images to the reusable portion during the first stage of the medical procedure; (h) the second pathway can comprise electrical connectors in the hub and in the reusable portion operatively mating with each other during said second stage of the medical procedure to thereby supply power from the reusable portion to the second imaging module and to convey the second images from the second imaging module to the reusable portion during the second stage of the medical procedure; (i) the first handle can be pencil-shaped and sized to be hand-held and can extend along a long axis of said first cannula; and (j) said first cannula can be straight.

According to some embodiments, a sterile kit for performing minimally invasive percutaneous nephrolithotomy (PCNL) comprises a first single-use portion and a second single-use portion configured for use in a single PCNL medical procedure on a patient, wherein: the first single-use portion comprises: a first handle; a first cannula that extends distally from a distal end of the first handle, has a sharp distal end, and is made of a material that is sufficiently stiff to be forced through the patient's skin and soft tissue to reach a kidney and/or ureter of the patient; a first channel that extends from a first proximal port at the first handle to a first distal port at the distal end of the first cannula and configured for passage of a guidewire; a first imaging module at the distal end of the first cannula comprising a first light source and a first image sensor configured to take first images of the patient's kidney and/or ureter and a portion of the guidewire protruding distally from the distal end of the first cannula; a first pathway configured to output the first images; the second single-use portion comprises: a hub; a second cannula that extends distally from a distal end of the fluid hub and is configured for insertion in the patient to reach the kidney and/or the ureter thereof after the first cannula has been withdrawn from the patient; wherein said second cannula has a second imaging module at a distal end thereof comprising a second light source and a second image sensor configured to take second images of the kidney and/or ureter; a second pathway configured to output the second images; wherein said second cannula is configured to rotate about a long axis thereof relative to said hub and has a flexible distal portion configured to bend away from said long axis to thereby enable taking said second images from different angles relative to the kidney and/or ureter; a force transfer mechanism configured to selectively bend said distal portion of the second cannula in response to user inputs at a proximal portion of the hub; and a sterile package that encloses the first and second single-use portions in a sterile environment for shipping the kit to a user and for storing the kit until needed for a PCNL procedure.

According to some embodiments, the kit described in the immediately preceding paragraph can further include one or more of the following: (a) the kit can further comprise a reusable portion, wherein the reusable portion comprises inputs for receiving said first images from the first single-use portion over said first pathway and for receiving said second images from said second reusable portion over the second pathway, electronics configured to process said first and second images into display images, a second handle and a display mounted thereon and configured to selectively display the display images, and controls configured to control said first and second imaging modules; (b) said inputs for receiving said first and second images can comprise a first connector for receiving the first images and a second connector, spaced from the first connector, for receiving the second images; (c) the reusable portion can include an electric motor operatively coupled with said flexible distal portion of the second cannula to bend said flexible portion under finger control by a user holding said reusable portion; (d) the kit can further include a sterile drape configured to cover said reusable portion while used in said medical procedure; (e) said first pathway can comprise a cable operatively coupled with said first imaging module, extending from the proximal end of the first handle, and releasably coupled to said reusable portion to thereby convey said first images to the reusable portion; (f) said first pathway can comprise a wireless connection between said first imaging module and said reusable portion configured to convey said first images to the reusable portion; (g) the first handle can be pencil-shaped and can extend along a long axis of said first cannula; and (h) said first cannula can be straight.

According to some embodiments, a kit for use in sialendoscopy comprises; a hub, a cannula extending distally from a distal end of the hub, a proximal device port and a proximal fluid port at a proximal end of the hub, a device distal port and a fluid distal port at a distal end of the cannula, an internal device channel between the device proximal and distal ports, an internal fluid channel between the proximal and distal fluid ports, and an imaging module at the distal end of the cannula; an external processor/display and a pathway thereto from the imaging module, said processor and display configured to receive images taken with said imaging module, process them into display images and selectively display the display images; wherein said pathway comprises at least one of (i) a cable operatively coupled with said imaging module and terminating in a connector and a connector at the image processor/display, and (ii) a wireless connection 1320 between the imaging module and the processor/display; wherein the cannula has an outside diameter no greater than 2 mm and the device channel has an inside diameter of no less than 0.8 mm.

According to some embodiments, the kit described in the immediately preceding paragraph can further include one or more of the following: (a) a two-dimensional image sensor and a light source in the imaging module; and (b) an LED source in the handle and one or more light fibers extending from the LED source to the distal end of the cannula.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through the accompanying drawings in which:

FIG. 1 is a perspective view of a first single-use portion of an endoscope for use in a first stage of a PCNL medical procedure, according to some embodiments.

FIG. 2 is a front view of a distal end of a first single-use portion, according to some embodiments.

FIG. 3 shows in perspective a first single-use portion for use in a first stage of a PCBL procedure and two views of a reusable portion for use in first and second stages of a PCNL medical procedure, according to some embodiments.

FIG. 4 illustrates a first single-use portion that has been inserted percutaneously to a patient's kidney and/or ureter in a first stage of a PCNL medical procedure, according to some embodiments.

FIG. 5 is otherwise like FIG. 4 but shows a first single-use portion connected with a reusable portion during a first stage of a PCNL medical procedure, according to some embodiments.

FIG. 6 is otherwise like FIG. 4 but shows a guidewire inserted into a channel of a first single-use portion during a first stage of the PCNL medical procedure, according to some embodiments.

FIG. 7 illustrates a second single-use portion assembled with a reusable portion into a hand-held endoscope as used in a second stage of a PCNL medical procedure, according to some embodiments.

FIG. 8 is a side view of a second single-use portion assembled with a reusable portion into a hand-held endoscope for use in a second stage of a PCNL medical procedure, according to some embodiments.

FIGS. 9 and 10 are perspective views, from different viewpoints, of a second single-use portion assembled and a reusable portion that mate into a hand-held endoscope for use in a second stage of a PCNL medical procedure, according to some embodiments.

FIG. 11 illustrates in perspective a sterile package enclosing a first single-use portion and a second single-use portion and, optionally, a drape for a reusable portion, according to some embodiments.

FIG. 12 illustrates reusable portion enclosed on all sides in a robe that can be sterile and can remain during a PCNL medical procedure, according to some embodiments.

FIG. 13 is a partly perspective and partly schematic view of kit for sialendoscopy, according to some embodiments.

FIG. 14 is a perspective view of a distal portion of cannula for sialendoscopy, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features. Like reference numbers and designations in the various drawings indicate like elements.

As described in more detail below, a sterile kit for a PCNL procedure comprises a first single-use portion and a second single-use portion, supplied in a sealed, sterile package. In a first stage of a PCNL medical procedure, the user unpacks the first single-use portion, penetrates a patient's skin with a sharp end of a first cannula and positions the tip of the first cannula at a desired location relative to the patient's kidney or ureter, typically while observing the first cannula's progress in the patient's body with an imaging modality such as ultrasound or fluoroscopy. Still in the first stage of the procedure, when the first cannula is at a desired position in the patient, the user threads a guidewire through a channel in the first single-use portion. The user then withdraws the first cannula from the patient, while keeping the guidewire in place in the patient, and inserts a trocar or another tube to a working area in the kidney or ureter. Before inserting the trocar, the passageway to the kidney may be enlarged with one or more dilators. In a second stage of the medical procedure, the user performs a medical procedure on the kidney and/or ureter, such as kidney stone extraction, using surgical instruments passed through the trocar and inserts, typically through the trocar, a second single-use portion assembled with a reusable portion into a hand-held endoscope, to view the work area during or after the surgical procedure.

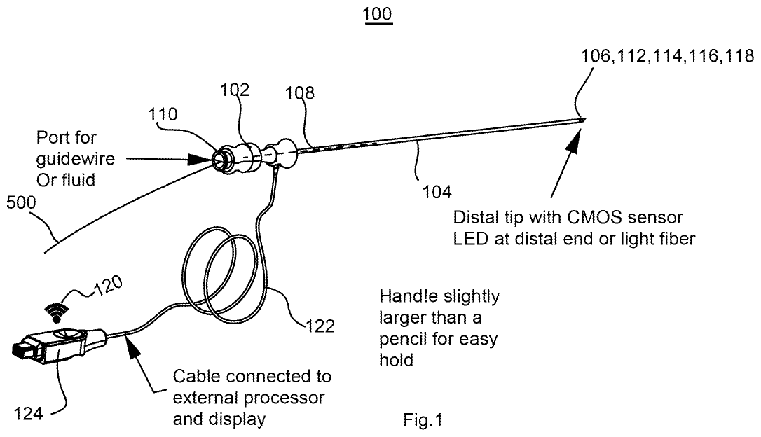

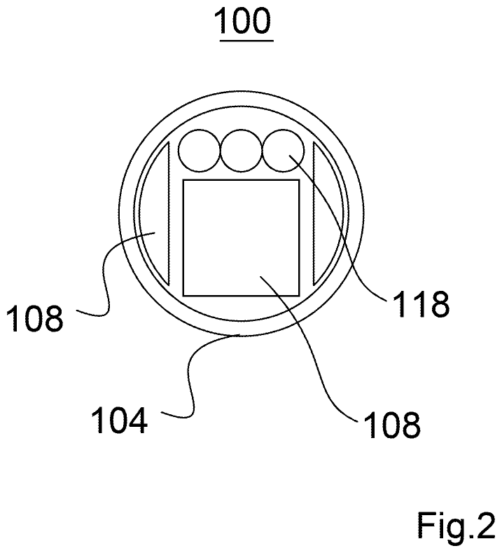

FIG. 1 is a perspective views of a first single-use portion 100 of an endoscope and FIG. 2 is a front view of a distal end of a first single-use portion, for use in a first stage of a minimally invasive percutaneous nephrolithotomy (PCNL) medical procedure, according to some embodiments. Single-use portion 100 comprises a first handle 102 that is shaped and dimensioned for holding by a user, typically with the user's fingers. In one example, first handle 102 is slightly thicker that a typical pencil. A first cannula 104 extends distally from a distal end of the first handle 102, has a sharp distal end 106, and is made of a material such as medical grade stainless steel that is sufficiently stiff to be forced through the skin and tissue of a patient to reach a kidney and/or ureter. The first single-use portion 100 is configured for insertion of a guidewire 500 through a proximal port 110 to extend distally through a first channel 108 and protrude if desired from a distal port 112. First single-use portion 100 has a first imaging module 114 at a distal end of the first cannula 104 that comprises a first light source 116 and a first two-dimensional image sensor 118, configured to take first images of a patient's kidney and/or ureter. The first images may include a portion of guidewire 500 that may protrude from distal end 106 of the first cannula 104. First single-use portion 100 further includes a first pathway operatively coupled with the first imaging module 114 and configured to output said first images. In one example, the first pathway comprises a cable 122 that outputs the first images through a connector 124. In another example, the first pathway comprises a wireless connection 120 such a WiFi connection that outputs the first images. WiFi sender 120 can be in connector 124 or in first handle 102. In a preferred embodiment, the working length of cannula 104 is 10 cm but other working lengths can be used for different patients or procedures. Channel 108 and ports 110 and 112 can be used for fluids instead of or in addition to being used for guidewire 500. First light source 116 can comprise one or more mini-LEDs at the distal end of cannula 104 or an LED source in handle 102 and one or more light fibers extending therefrom to the distal end of first cannula 104.

FIG. 2 is a front view of a distal end of first cannula 104, according to some embodiments, and illustrates one example of arranging the LEDs or light fibers 118, image sensor 108, and channel 108 (which in this example can be two parallel channels) inside first cannula 104.

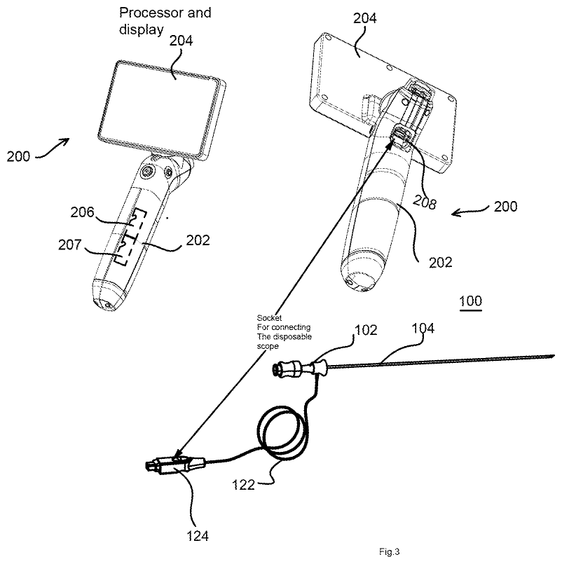

FIG. 3 shows first single-use portion 100 and two perspective views from different angles of a reusable portion 200 that releasably couples with the first single-use portion 100 during a first stage of a PCNL medical procedure to form an assembled endoscope, according to some embodiments. Reusable portion 200 comprises a second handle 202, a display 204, electronics 206 and, optionally, a battery 207 that can be entirely or partly inside second handle 202 and/or display 204, and a connector such as a socket 208 configured to mate with connector 124 of the first single-use portion.

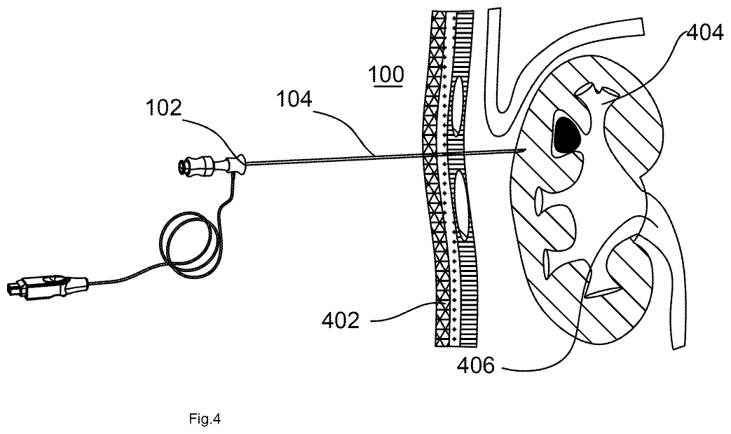

FIG. 4 illustrates use of single-use portion 100 during a first stage of a PCNL medical procedure, in which cannula 104 of single-use portion 100 is inserted through the skin and into tissue of a patient 402 to or into the patient's kidney 404 and/or ureter 406, for example to the location of a kidney stone shown in black. This typically is done under guidance of an imaging modality such as ultrasound or fluoroscopy. For at least a part of this insertion, single-use portion 100 may but need not be assembled with reusable portion 200 into an endoscope. Display 204 can show first images taken with first imaging module 114. Instead, or in addition, the first images can be sent to and shown on a separate display, not shown, via a cable of wireless connection 120.

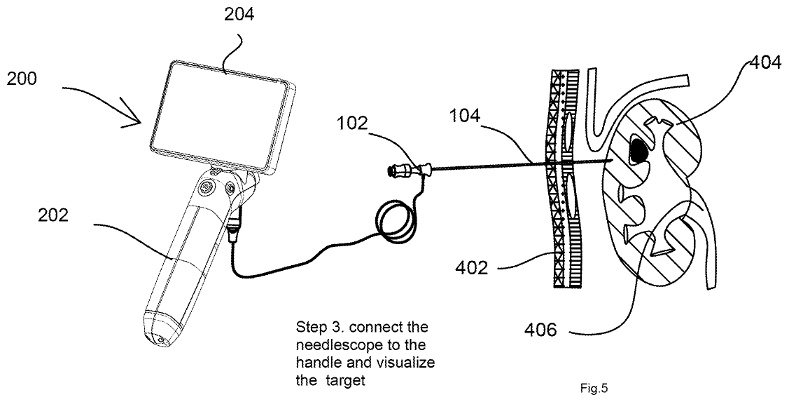

FIG. 5 illustrates single-use portion 100 coupled with reusable portion 200 through cable 122 (and connector 124 and socket 208 seen in FIG. 3) during at least a part of the first stage of the PCNL medical procedure. Preferably, for at least a terminal part of the insertion, the single-use portion 100 and reusable portion 200 are coupled so that first images from imaging module 114 are transmitted to and displayed on display 204. While first image module 114 is inside kidney 404 and/or ureter 406, a user can carry out fine positioning using first handle 102 while observing the first images on display 204, until the distal end 106 of first cannula 104 is at a desired position in kidney 406 and/or ureter 406.

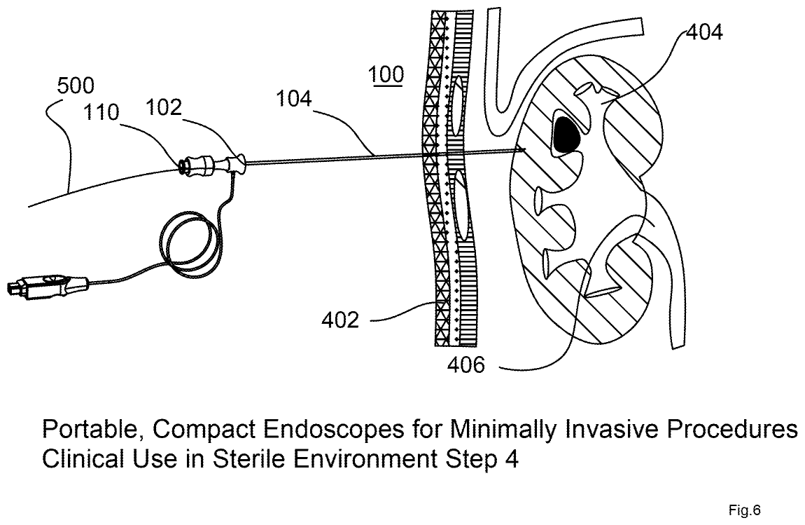

FIG. 6 illustrates threading a guidewire 500 through port 110 until the guidewire is at or protrudes through a distal port 112 that now is at the desired position in kidney 404 and/or ureter 406. The medical professional typically threads guidewire 500 until a desired portion thereof protrudes distally from distal port 106 of first cannula 104. While not seen in FIG. 6, reusable portion 200 may remain coupled with single-use portion 100 while guidewire 500 is being threaded through (as seen in FIG. 5) so the user can observe a portion of guidewire 500 that protrudes from first cannula 104 as guidewire 500 is being threaded through, to thereby assist in fine positioning the distal end of the guidewire.

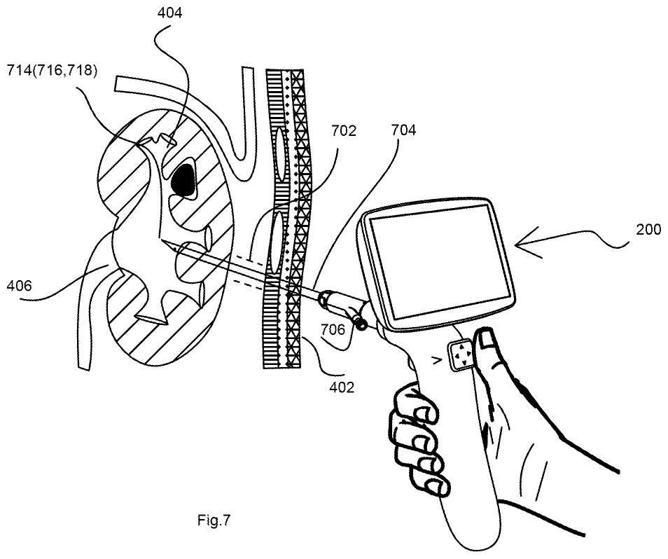

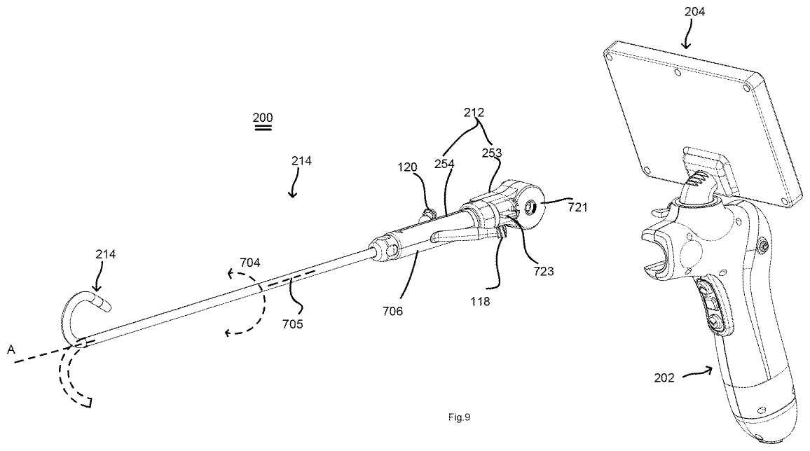

FIG. 7 illustrates an example of a second stage of a PCNL medical procedure. After the first stage described above is completed, the user withdraws single-use portion 100 from patient 402 over guidewire 500, leaving the guidewire in place in the patient, and in one example inserts one or more dilators to enlarge a path to the kidney and then positions a trocar 702 or another tube into the patient over guidewire 500. The user performs a medical procedure to treat kidney 404 and/or ureter 406 through the trocar or tube 702, using instruments passed through the trocar or tube or through a channel in the second single-use portion. In one example, the outside diameter of second cannula 704 is 4.5 mm or less and the inside diameter of a channel 705 (FIG. 9) in the second single-use portion is 2 mm. Other dimensions are possible.

During and after treating the work area in the second stage of the medical procedure, the user can observe the kidney and/or ureter or other nearby anatomy with a second imaging module 714 at a distal end of second cannula 706, which is configured to take and convey second images for display at display 204. For use in this second stage of PCNL, the user assembles an endoscope comprising a second single-use portion 700 that comprises a second cannula 704 and a hub 706 releasably coupled with single user portion 200 to form a hand-held endoscope. Second cannula 704 has at its distal end a second imaging module 714 that can be like first imaging module 114 and comprise a light source 716 and two-dimensional image sensor 718.

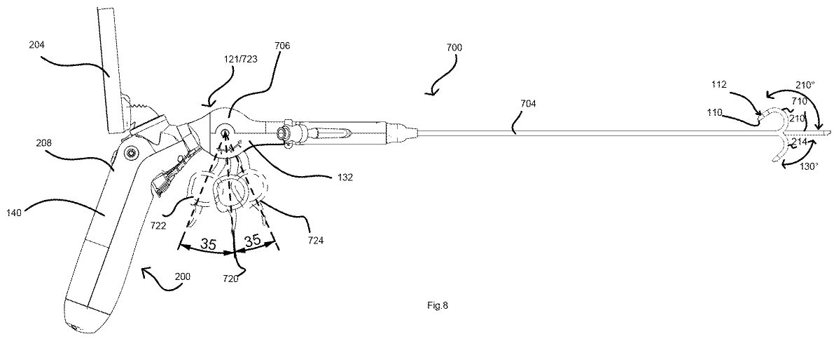

FIG. 8 illustrates in more detail an example of a second single-use portion 700 coupled with reusable portion 200. Details of portions 700 and 200 can be like those of portions 104 and 102, respectively, described in prior U.S. patent application Ser. No. 17/370,575 filed Jul. 8, 2021 and published as US 2021/0338052 A1, except for the addition of socket 208 (FIG. 3). Said prior patent application is hereby incorporated by reference in this patent specification. Some reference numerals are those used in the subject patent specification. A more detailed description of the illustrated endoscope can be found in the prior application.

As seen in FIG. 8 and described in detail in said prior application Ser. No. 17/370,575, second single-use portion 700 comprises a hub 706 and a second cannula 704 (identified by reference numerals 120 and 170 in the Ser. No. 17/370,575 application). Second single-use portion 700 has a flexible distal portion 210 that bends away from the long axis of cannula 704, as illustrated and described in said application Ser. No. 17/370,575. The bending is controlled in this example by a finger lever 720, and single-use portion 704 couples with reusable portion 200 to form an endoscope as illustrated and described in said application Ser. No. 17/370,575. Imaging module 714 operatively couples with display 204, and finger-operated control 720 operatively couples with the flexible distal portion as described in the prior application, for example through cables.

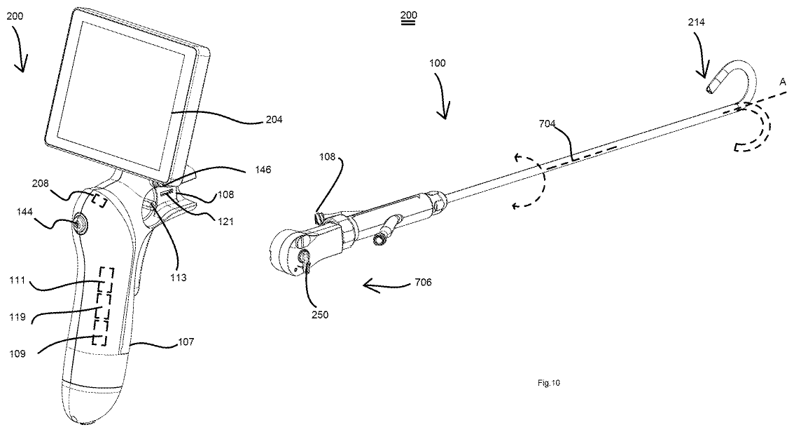

FIGS. 9 and 10 illustrate another example of a second single-use portion 700 that operatively couples with reusable portion 200 to form an endoscope for use in a second stage of a PCNL medical procedure. FIGS. 9 and 10 are otherwise like FIGS. 2B and 2C of prior U.S. patent application Ser. No. 18/083,209 filed Dec. 16, 2022, except for the addition of socket 208 and for showing several components from other figures of the prior application and for the addition of several reference numerals used in the subject patent specification. As described in said prior application Ser. No. 18/083,209, which is hereby incorporated by reference, reusable portion 200 includes an electric motor 111 and includes electronics 119 configured to process the second images into display images for displaying on display 204 and to control bending of the flexible distal portion of second cannula 704. Electronics 119 can be in handle 202 and/or in display 204. A power source such as battery 109 can power the electrical components. Electric motor 111 is operatively coupled with the flexible distal portion of second cannula 704 to bend that flexible portion in a selected direction and through a selected angle under manual control over the motor operation through a finger-operated button 144 so that the work area can be observed from different angles and the distal end of the second cannula can be directed as desired into anatomy such as the ureter. As illustrated in FIGS. 9 and 10 and described in said prior application Ser. No. 18/083,209, reusable portion 200 has a shaft 113 rotated by motor 111 to drive a gear 721 in hub 706 and thus selectively bend the distal portion of cannula 704 (206). As seen in FIGS. 9 and 10 and described in said prior application, proximal ports 118, 120 can be used for surgical instruments and/or fluid inputs and outputs, and one or more distal ports are provided at the distal end of second cannula 700. As illustrated in FIGS. 9 and 10 and described in said prior application, the second cannula 700 is configured to rotate about its long axis relative to reusable portion 200 and preferably relative to hub 706, whereby the combination of bending the distal flexible portion of second cannula 704 and rotating cannula 704 enable the user to view the work area and adjacent anatomy from different viewpoints.

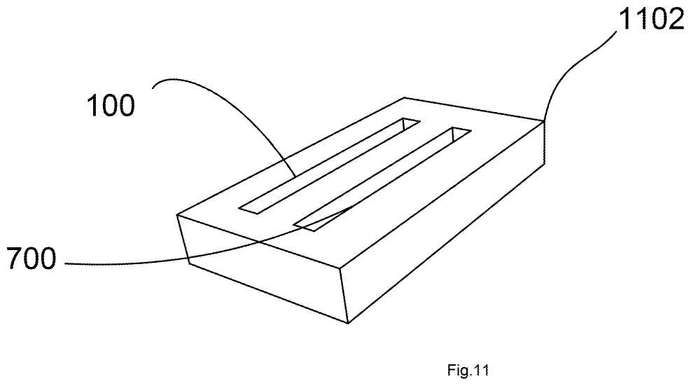

FIG. 11 schematically illustrates a sterile package 1102 enclosing the first and second single use-portions 100 and 700 in a sterile environment as a kit for shipping to users and for storing in a sterile environment until needed for a PCNL medical procedure.

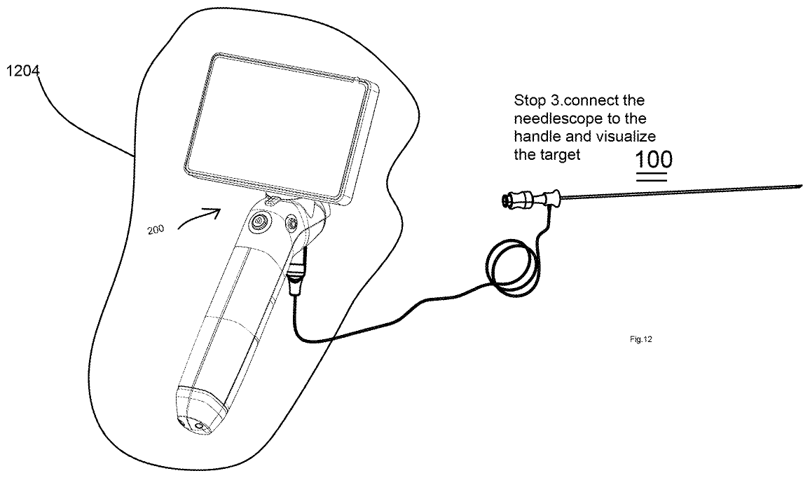

FIG. 12 illustrates reusable portion 200 enclosed on all sides in a robe 1202 that can be sterile and can remain over portion 200 while portion 200 is coupled with single-use portion 100 during the first stage of the PCNL medical procedure and/or while single-use portion 700 is coupled with reusable portion 200 during the second stage of the medical procedure. In some but not all examples, robe 1202 may be included in sterile package 1102 and kept there in a sterile environment.

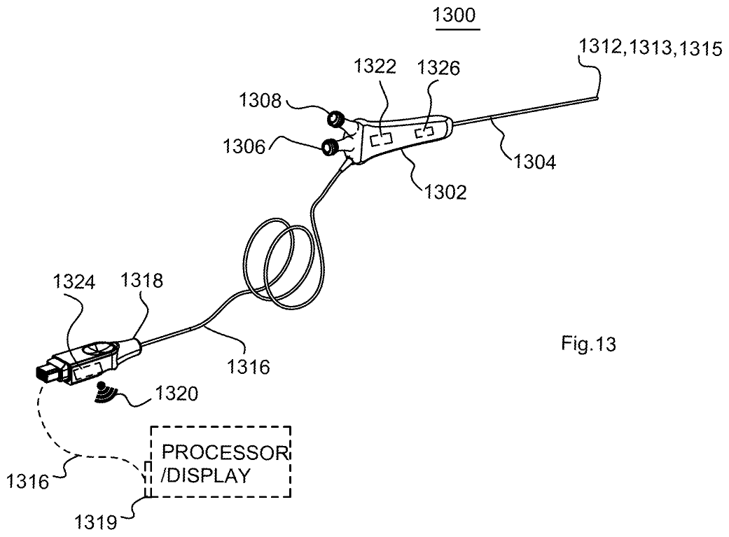

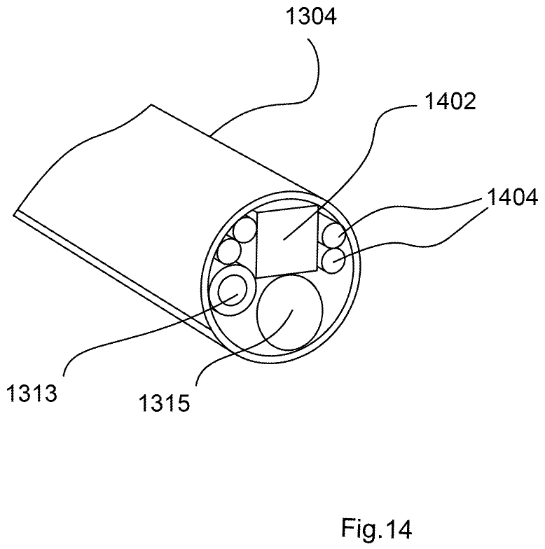

FIG. 13 is a partly perspective and partly schematic view of kit 1300 for sialendoscopy, according to some embodiments. The kit comprises a hub 1302 configured for grasping by a user's hand and a cannula 1304 extending distally from a distal end of the hub. A proximal device port 1306 and a proximal fluid port 1308 are at a proximal end of the hub, and a device distal port 1312 and a fluid distal port 1313 are at a distal end of the cannula. An internal device channel 1315 between the device proximal and distal ports and an internal fluid channel 1313 between the proximal and distal fluid ports extend through handle 1302 and cannula 1304. An imaging module 1312 is at the distal end of cannula 1304. An external processor/display 1314 is operatively coupled with imaging module 1312 through a pathway that can be (i) a cable 1316 terminating in a connector 1318 releasably mating with a connector 1319 at processor display 1314, and/or (ii) a wireless connection 1320 that can include a transceiver 1322 in handle 1318 or 1324 in connector 1318.

As shown, the distal end of cannula 1304 houses device distal port 1313 and fluid distal port 1315, and two-dimensional image sensor 1402 that together with light source 1404 forms imaging module 1312. Light source 1404 can comprise one or more mini-LEDSs at the distal end of cannula 1304 or one or more light fibers operatively coupled with an LED source 1326 in handle 1302. In a non-limiting example, the outside diameter of cannula 1302 is 1.55 mm, device channel 1315 has an inside diameter 0.8 mm, fluid channel 1313 has inside diameter 0.3 mm, each light fiber 1404 had diameter 0.25 mm, and image sensor 1402 is a square 0.64 mm per side. Kit 300 preferably is supplied to a user in a sterile package like that shown in FIG. 11 but with a single compartment. Processor/display 1314 is not in the sterile package.

In operation, the user removes kit 1300 from the sterile package and enables pathway 1316 or 1320 to establish communication between image module 1312 and processor/display 1314. Controls over operations of imaging module 1312, such as for taking still or video images, can be provided on handle 1302 and/or at other locations such as at processor/display 1314 or in floor switches. Guided by images taken with imaging module 1312 and displayed at processor/display 1314, the user advances the distal end of cannula 1304 in a salivary duct to a desired location in the duct or at a salivary gland. The user can connect a fluid source to proximal fluid port 1308 to flush tissue or deliver medication, and can pass surgical implements such as ablation needles or graspers through proximal device port 1306 to and out of distal device port 1315 to perform medical procedures while observing the work area at processor/display 1304.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. A kit for performing minimally invasive percutaneous nephrolithotomy (PCNL) medical procedure, comprising:
a first single-use portion 100 comprising a first handle 102, a first cannula 104 that extends distally from the first handle and has a sharpened distal end 106, a first channel 108 that extends from a first proximal port 110 in the handle to a first distal port 112 in the cannula and is configured for insertion of guidewire 500 through and distally out of the first channel, and a first imaging module 114 that is at a distal end of the first cannula and comprises a first light source 116 and a first image sensor 118;
a second single-use portion 700 comprising a hub 706, a second cannula 704 that extends distally from the hub, and a second imaging module 714 that is a distal end of the second cannula and comprises a second light source 716 and a second image sensor 718;
wherein the first cannula is configured for insertion to a patient's kidney 404 and/or ureter 406 and the first imaging module is configured to take first images thereof that selectively include any guidewire portion extending out of the distal end of the first channel during a first stage of the medical procedure;
wherein the second cannula is configured for insertion to the patient's kidney and/or ureter during a second stage of the medical procedure, after the first canula has been withdrawn from the patient, and the second imaging module is configured to take second images thereof during a second stage of the medical procedure;
a reusable portion 200 that comprises a second handle 202, a display 204 carried thereby and image-processing electronics 206;
wherein the reusable portion is configured to couple with the first single-use portion during said first stage of the medical procedure to thereby receive said first images and process and display them, and to couple with said second single-use portion during said second stage of the medical procedure to thereby receive said second images and process and display them;
wherein said kit further includes a first pathway 120, 122-124 configured to convey said first images from the first imaging module to the reusable portion during the first stage of the medical procedure and a second pathway 721, 121-123 configured to convey the second images from the second imaging module to the reusable portion during in second stage of the medical procedure.

2. The kit of claim 1, further comprising a sterile package 1102 enclosing said first and second single-use portions 100 and 700, respectively, for shipping to a user as a sterile kit and for storing as a sterile kit until needed for a PCNL procedure.

3. The kit of claim 1, in which the second cannula includes a flexible distal portion 210, 214 configured to bend away from a long axis and the second cannula and the reusable portion includes an electrical motor 111 operatively coupled with said flexible portion to bend the flexible portion in a selected direction and through a selected angle under user control over the motor operation.

4. The kit of claim 3, in which the reusable portion has a side opening with a shaft 113 extending transversely to the long axis and configured to be rotated by said motor, and said hub includes a gear 721 mating with said shaft during said second stage of the medical procedure.

5. The kit of claim 1, in which the hub includes a finger control 720 operatively coupled with said flexible portion and configured to bend the flexible portion in a selected direction and through a selected angle in response to finger force exerted on the finger control.

6. The kit of claim 1, in which the first single-use portion is configured for withdrawal from the patient over the guidewire after the first stage of the medical procedure, leaving the guidewire in the patient, and further including a trocar 702 configured to the inserted in the patient to the patient's kidney over the guidewire, enabling surgical instruments to be inserted through the trocar to the kidney and/or ureter.

7. The kit of claim 1, in which said first pathway comprises a cable 122 operatively coupled with said first imaging module, extending from the first handle and terminating in a first connector 124 and the reusable portion comprises a matching connector 208 configured to make electrical contact with the first connector and to convey said first images to the reusable portion.

8. The kit of claim 1, in which said first pathway comprises a wireless connection 120 between said first imaging module and said reusable portion configured to convey said first images to the reusable portion during the first stage of the medical procedure.

9. The kit of claim 1, in which the second pathway comprises electrical connectors 121, 723 in the hub and 108 in the reusable portion operatively mating with each other during said second stage of the medical procedure to thereby supply power from the reusable portion to the second imaging module and to convey the second images from the second imaging module to the reusable portion during the second stage of the medical procedure.

10. The kit of claim 1, in which the first handle is sized for hand-holding.

11. The kit of claim 10, in which said first cannula is straight.

12. A sterile kit for performing minimally invasive percutaneous nephrolithotomy (PCNL) comprising a first single-use portion 100 and a second single-use portion 700 configured for use in a single PCNL medical procedure on a patient 402, wherein:
the first single-use portion 100 comprises:
a first handle 104;
a first cannula 102 that extends distally from a distal end 106 of the first handle, has a sharp distal end, and is made of a material that is sufficiently stiff to be forced through the patient's skin and soft tissue to reach a kidney 404 and/or ureter 406 of the patient;
a first channel 108 that extends from a first proximal port 110 at the first handle to a first distal port 112 at the distal end of the first cannula and configured for passage of a guidewire 500;
a first imaging module 114 at the distal end of the first cannula comprising a first light source 116 and a first image sensor 118 configured to take first images of the patient's kidney and/or ureter and a portion of the guidewire protruding distally from the distal end of the first cannula;
a first pathway 120, 122-124 configured to output the first images;
the second single-use portion 700 comprises:
a hub 706;
a second cannula 704 that extends distally from a distal end of the fluid hub and is configured for insertion in the patient to reach the kidney and/or the ureter thereof after the first cannula has been withdrawn from the patient;
wherein said second cannula has a second imaging module 714 at a distal end 214 thereof comprising a second light source 716 and a second image sensor 718 configured to take second images of the kidney and/or ureter;
a second pathway 108, 121, 723 configured to output the second images;
wherein said second cannula is configured to rotate about a long axis thereof relative to said hub and has a flexible distal portion 214 configured to bend away from said long axis to thereby enable taking said second images from different angles relative to the kidney and/or ureter;
a force transfer mechanism 720,113-721 configured to selectively bend said distal portion of the second cannula in response to user inputs at a proximal portion of the hub; and
a sterile package 1102 that encloses the first and second single-use portions in a sterile environment for shipping the kit to a user and for storing the kit until needed for a PCNL procedure.

13. The sterile kit of claim 12, further comprising a reusable portion 200, wherein the reusable portion comprises inputs 208,121 for receiving said first images from the first single-use portion over said first pathway and for receiving said second images from said second reusable portion over said second pathway, electronics 206, 119 configured to process said first and second images into display images, a second handle 202 and a display 204 mounted thereon and configured to selectively display the display images, and controls 720, 144 configured to control said first and second imaging modules.

14. The sterile kit of claim 13, in which said inputs for receiving said first and second images comprise a first connector 208 for receiving the first images and a second connector 121, spaced from the first connector, for receiving the second images.

15. The sterile kit of claim 13, in which the reusable portion includes an electric motor 111 operatively coupled with said flexible distal portion of the second cannula to bend said flexible portion under finger control by a user holding said reusable portion.

16. The sterile kit of claim 13, further including a sterile drape 1204 configured to cover said reusable portion while used in said medical procedure.

17. The sterile kit of claim 12, in which said first pathway comprises a cable 122 operatively coupled with said first imaging module, extending from the proximal end of the first handle, and releasably coupled to said reusable portion to thereby convey said first images to the reusable portion.

18. The sterile kit of claim 12, in which said first pathway comprises a wireless connection 120 between said first imaging module and said reusable portion configured to convey said first images to the reusable portion.

19. The sterile kit of claim 12, in which the first handle 102 is configured to be held by a user's fingers.

20. The sterile kit of claim 12, in which said first cannula 104 is straight.

21. A single-use kit for sialendoscopy, comprising:
a hub 1302, a cannula 1304 extending distally from a distal end of the hub, a proximal device port 1306 and a proximal fluid port 1308 at a proximal end of the hub, a device distal port 1312 and a fluid distal port 1313 at a distal end of the cannula, an internal device channel 1315 between the device proximal and distal ports, an internal fluid channel 1313 between the proximal and distal fluid ports, and an imaging module 1312 at the distal end of the cannula;
an external processor/display 1314 and a pathway 1316, 1320 thereto from the imaging module, said processor and display configured to receive images taken with said imaging module, process them into display images and selectively display the display images;
wherein said pathway comprises at least one of (i) a cable 1316 operatively coupled with said imaging module and terminating in a connector 1318 and a connector 1319 at the image processor/display, and (ii) a wireless connection 1320 between the imaging module and the processor/display;
wherein the cannula has an outside diameter no greater than 2 mm and the device channel has an inside diameter of no less than 0.8 mm.

22. The single-use kit for sialendoscopy of claim 21, in which the imaging module comprises a two-dimensional image sensor 1402 and a light source 1404.

23. The single-use kit for sialendoscopy of claim 22, in which the light source comprises an LED source 1322 in the handle and one or more light fibers 1404 extending from the LED source to the distal end of the cannula.
